(19) United States
(12) Patent Application Publication
(10) Pub. No.: US 2022/0280336 A1
(43) Pub. Date: Sep. 8, 2022

(54) Indicators for Confirming Fluid Connections in Systems, Pads, and Methods for Targeted Temperature Management

(71) Applicant: C. R. Bard, Inc., Franklin Lakes, NJ (US)

(72) Inventors: Jesse Smith, Broomfield, CO (US); Mengjia Yi, Westminster, CO (US); Niharika Pathare, Thane (IN); Gabriel A. Johnston, Broomfield, CO (US)

(21) Appl. No.: 17/689,791

(22) Filed: Mar. 8, 2022

Related U.S. Application Data
(60) Provisional application No. 63/158,277, filed on Mar. 8, 2021.

(51) Int. Cl. A61F 7/02; A61F 7/00

(52) U.S. Cl. CPC A61F 7/02; A61F 7/0085; A61F 2007/0054; A61F 2007/0215; A61F 2007/0226

(57) ABSTRACT

Disclosed are indicators for confirming fluid connections in systems, pads, and methods thereof for targeted temperature management. For example, a pad can include a multilayered pad body, a pad inlet connector, a pad outlet connector, and an indicator for visual indication of charging a conduit layer of the pad body with a supply fluid and confirmation of accurate fluid connections. The conduit layer includes one or more conduits for conveying a temperature-controlled fluid as the supply fluid from a hydraulic system of a control module as well as convey a return fluid back to the hydraulic system. A thermally conductive adhesive layer over the conduit layer is for placement on a portion of a patient's body. The pad inlet connector includes a pad inlet for charging the conduit layer with the supply fluid. The pad outlet connector includes a pad outlet for discharging the return fluid from the conduit layer.

### PRIORITY

This application claims the benefit of priority to U.S. Provisional Patent Application No. 63/158,277, filed Mar. 8, 2021, which is incorporated by reference in its entirety into this application.

### BACKGROUND

Targeted temperature management (“TTM”) is a treatment for maintaining a therapeutic body temperature (e.g., hypothermia) in a patient for a period of time to improve the patient's outcome in any of a number of different medical situations. Any of a number of current systems for TTM use a number of fluid delivery lines to convey temperature-controlled fluid from a control module to one or more pads placed on one or more portions of the patient's body. With the number fluid delivery lines and connectors thereof that can be required in a given medical situation, it can be difficult to quickly and accurately fluidly connect the number of fluid delivery lines together, particularly for an inexperienced clinician under a time constraints. What is needed are indicators for confirming fluid delivery lines are accurately connected in systems, pads, and methods for TTM.

Disclosed herein are indicators for confirming fluid connections in systems, pads, and methods thereof for TTM.

### SUMMARY

Disclosed herein is a pad for TTM including, in some embodiments, a multilayered pad body, a pad inlet connector, a pad outlet connector, and an indicator configured to visually indicate the charging of a conduit layer with a supply fluid for confirmation of an accurate fluid connection. The pad body includes the conduit layer and a thermally conductive adhesive layer over the conduit layer. The conduit layer includes one or more conduits. The one-or-more conduits are configured to convey a temperature-controlled fluid as the supply fluid from a hydraulic system of a control module. The one-or-more conduits are also configured to convey a return fluid back to the hydraulic system. The adhesive layer is configured for placement on a portion of a patient's body. The pad inlet connector includes a pad inlet configured for charging the conduit layer with the supply fluid. The pad outlet connector includes a pad outlet configured for discharging the return fluid from the conduit layer.

In some embodiments, the pad body further includes one or more translucent windows. The one-or-more translucent windows enable visualization of the indicator with the charging of the conduit layer with the supply fluid.

In some embodiments, the one-or-more translucent windows are about the pad inlet connector, the pad outlet connector, or both the pad inlet connector and the pad outlet connector.

In some embodiments, the pad body is of a translucent elastomer. The translucent elastomer enables visualization of the indicator with the charging of the conduit layer with the supply fluid.

In some embodiments, the pad further includes a secondary fluid-delivery line (“FDL”). The secondary FDL is configured to convey the supply fluid from the hydraulic system. The secondary FDL is also configured to convey the return fluid back to the hydraulic system. The secondary FDL is split at a pad-connecting end of the secondary FDL. The pad-connecting end of the secondary FDL includes a pair of secondary FDL connectors including a secondary FDL outlet connector and a secondary FDL inlet connector. The secondary FDL outlet connector is configured to fluidly connect to the pad inlet connector. The secondary FDL inlet connector is configured to fluidly connect to the pad outlet connector.

In some embodiments, at least a portion of the secondary FDL is translucent. The translucent portion of the secondary FDL enables visualization of the indicator with the charging of the conduit layer with the supply fluid.

In some embodiments, one-or-more connectors of the pad inlet connector, the pad outlet connector, the secondary FDL outlet connector, and the secondary FDL inlet connector include a translucent portion, thereby enabling visualization of the indicator with the charging of the conduit layer with the supply fluid.

In some embodiments, the indicator is a chemical indicator disposed in the one-or-more conduits or integrated into the conduit layer.

In some embodiments, the chemical indicator is CoCl₂ disposed in the one-or-more conduits. The CoCl₂ is provided in an amount sufficient to impart a discernable pink color to the supply fluid when solvated in the supply fluid with the charging of the conduit layer with the supply fluid.

In some embodiments, the chemical indicator is a fluorophore disposed in the one-or-more conduits. The fluorophore is provided in an amount sufficient to impart a discernable fluorescence to the supply fluid when solvated in the supply fluid with the charging of the conduit layer with the supply fluid.

In some embodiments, the chemical indicator is a fluorophore covalently bonded to a polymer forming the conduit layer. The fluorophore is provided in an amount sufficient to impart a discernable fluorescence to the supply fluid when partially solvated in the supply fluid with the charging of the conduit layer with the supply fluid.

In some embodiments, the fluorophore absorbs light about an excitation wavelength and emits light about an emission wavelength different than the absorption wavelength. At least the emission wavelength is in a visible portion of the electromagnetic spectrum.

In some embodiments, the fluorophore exhibits solvatochromism in accordance with a composition of the supply fluid. The supply fluid is water, ethylene glycol, or a mixture thereof.

In some embodiments, at least the secondary FDL outlet connector includes an integrated light-emitting diode (“LED”) module. The LED module includes one or more LEDs configured to illuminate the supply fluid as it flows through the secondary FDL outlet connector.

In some embodiments, the LED module includes a water turbine and an electrical generator. The water turbine is configured to rotate a rotor of the electrical generator relative to a stator of the electrical generator to produce electrical energy to illuminate the supply fluid with the one-or-more LEDs.

In some embodiments, the LED module includes a temperature sensor and LED-illuminating logic. The LED-illuminating logic is configured to selectively illuminate one LED of the one-or-more LEDs in accordance with a predetermined temperature range. Each LED of the one-or-more LEDs is configured to illuminate the supply fluid with a different color.

In some embodiments, at least the secondary FDL outlet connector includes an integrated laser diode module. The laser diode module includes one or more laser diodes configured to illuminate the supply fluid as it flows through the secondary FDL outlet connector. The one-or-more conduits are configured to promote internal reflection of light and, thereby, illuminate an entirety of the conduit layer.

In some embodiments, the pad further includes an impermeable film between the conduit layer and the adhesive layer. The impermeable film is configured to retain the supply fluid in the conduit layer.

In some embodiments, the adhesive layer includes a hydrogel. The hydrogel is selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

In some embodiments, the pad of further includes a release liner over the adhesive layer in a ready-to-use state of the pad. The release liner is configured to maintain integrity of at least the adhesive layer prior to use of the pad.

Also disclosed herein is a system for TTM including, in some embodiments, a control module, a primary FDL, and one or more pads configured for placement on one-or-more portions of a patient's body, respectively. The control module includes a hydraulic system configured to provide a temperature-controlled fluid. The primary FDL is configured to convey the temperature-controlled fluid as a supply fluid from the hydraulic system. The primary FDL is also configured to convey a return fluid back to the hydraulic system. Each pad of the one-or-more pads includes a multilayered pad body, a pad inlet connector, a pad outlet connector, and an indicator configured to visually indicate the charging of a conduit layer with the temperature-controlled fluid for confirmation of an accurate fluid connection. The pad body includes the conduit layer, which, in turn, includes one or more conduits configured to convey the temperature-controlled fluid. The pad inlet connector includes a pad inlet configured for charging the conduit layer with the supply fluid. The pad outlet connector includes a pad outlet configured for discharging the return fluid from the conduit layer.

In some embodiments, the hydraulic system further includes a chiller evaporator, a heater, a hydraulic-system outlet, and a hydraulic-system inlet. The chiller evaporator is configured for fluid cooling. The heater is configured for fluid heating. The chiller evaporator and the heater, together, are configured to provide the temperature-controlled fluid. The hydraulic-system outlet is configured for discharging the supply fluid from the hydraulic system. The hydraulic-system inlet is configured for charging the hydraulic system with the return fluid to continue to produce the temperature-controlled fluid.

In some embodiments, the control module further includes one or more processors, primary memory, and instructions stored in the primary memory configured to instantiate one or more processes for TTM with the control module.

Also disclosed herein is a method of a system for TTM including, in some embodiments, a pad-placing step, a charging step, a confirming step, and a fluid-circulating step. The pad-placing step includes placing a pad on a portion of a patient's body with a thermally conductive adhesive layer of a multilayered pad body of the pad in contact with skin of the portion of the patient's body. The charging step includes charging a conduit layer of the pad with a supply fluid of a temperature-controlled fluid. The supply fluid is provided by a hydraulic system of a control module by way of a combination of fluidly connected FDLs including a secondary FDL and a primary FDL. The confirming step includes visually confirming a presence of an indicator configured to visually indicate the charging of the conduit layer with the supply fluid for confirmation of an accurate fluid connection. The fluid-circulating step includes circulating the supply fluid through a conduit layer of the pad body to cool or warm the portion of the patient's body as needed in accordance with TTM.

In some embodiments, the method further includes a pad-connecting step. The pad-connecting step includes fluidly connecting a secondary FDL outlet connector at a split pad-connecting end of the secondary FDL to a pad inlet connector. The pad-connecting step also includes fluidly connecting a secondary FDL inlet connector at the split pad-connecting end of the secondary FDL to a pad outlet connector.

In some embodiments, the confirming step includes confirming a color or fluorescence of a chemical indicator solvated in the supply fluid. Such confirming is through a translucent window of the pad body, a translucent polymer of the pad body, a translucent portion of the secondary FDL, a translucent portion of one or more connectors of the pad inlet connector, the pad outlet connector, the secondary FDL outlet connector, and the secondary FDL inlet connector.

In some embodiments, the confirming step includes confirming illumination of the supply fluid with an integrated LED module of at least the secondary FDL outlet connector through a translucent window of the pad body, a translucent polymer of the pad body, a translucent portion of the secondary FDL, a translucent portion of one or more connectors of the pad inlet connector, the pad outlet connector, the secondary FDL outlet connector, and the secondary FDL inlet connector.

In some embodiments, the method further includes another confirming step. The other confirming step includes visually confirming the supply fluid is within a particular temperature range. The LED module includes a temperature sensor and LED-illuminating logic configured to selectively illuminate one LED of one or more LEDs in accordance with a predetermined temperature range. Each LED of the one-or-more LEDs is configured to illuminate the supply fluid with a different color.

In some embodiments, the method further includes a release liner-removal step. The release liner-removal step includes removing a release liner of the pad to reveal the adhesive layer before the pad-placing step. The release liner is configured to maintain integrity of at least the adhesive layer prior to using the pad.

In some embodiments, the fluid-circulating step transfers heat between the supply fluid and the portion of the patient's body by thermal conduction through the adhesive layer.

In some embodiments, the fluid circulating step includes circulating a cool fluid through the conduit layer to bring the patient into hypothermia.

In some embodiments, the fluid circulating step includes circulating a warm fluid through the conduit layer to bring the patient into normothermia.

These and other features of the concepts provided herein will become more apparent to those of skill in the art in view of the accompanying drawings and following description, which describe particular embodiments of such concepts in greater detail.

### DRAWINGS

FIG. 1 illustrates a TTM system in accordance with some embodiments.

FIG. 2A illustrates left and right TTM pads for a torso of a patient in accordance with some embodiments.

FIG. 2B illustrates left and right TTM pads for legs of a patient in accordance with some embodiments.

FIG. 3 illustrates a multilayered pad body of a TTM pad in accordance with some embodiments.

FIG. 4 illustrates a fluorophore covalently bonded to a translucent polymer in accordance with some embodiments.

FIG. 5 illustrates a hydraulic system of a control module in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

Any of a number of current systems for TTM use a number of fluid delivery lines to convey temperature-controlled fluid from a control module to one or more pads placed on one or more portions of the patient's body. With the number fluid delivery lines and connectors thereof that can be required in a given medical situation, it can be difficult to quickly and accurately fluidly connect the number of fluid delivery lines together, particularly for an inexperienced clinician under a time constraints. What is needed are indicators for confirming fluid delivery lines are accurately connected in systems, pads, and methods for TTM.

Disclosed herein are indicators for confirming fluid connections in systems, pads, and methods thereof for TTM.

### TTM Systems

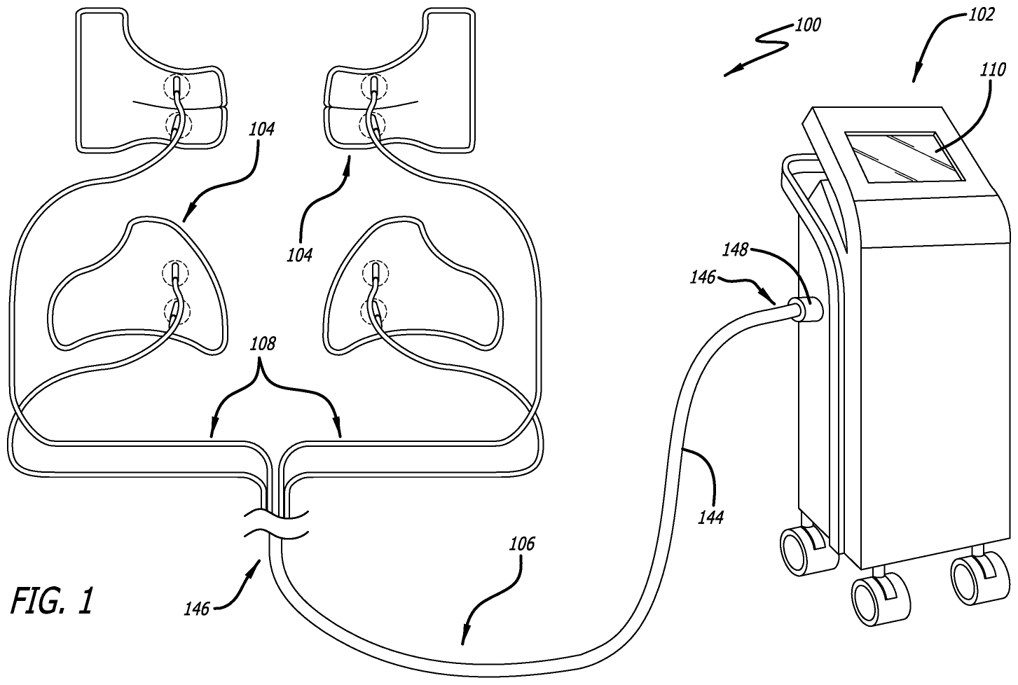

FIG. 1 illustrates a TTM system 100 in accordance with some embodiments.

As shown, the system 100 can include a control module 102, one or more TTM pads 104 such as those set forth below, a primary FDL 106, and one or more secondary FDLs 108 corresponding in number to the one-or-more pads 104. Description for the control module 102 is set forth immediately below. Description for the one-or-more pads 104 is set forth in the following section.

The control module 102 can include a console 110 with an integrated display screen configured as a touchscreen for operating the control module 102. The console 110 can include one or more processors, primary and secondary memory, and instructions stored in the primary memory configured to instantiate one or more processes for TTM with the control module 102.

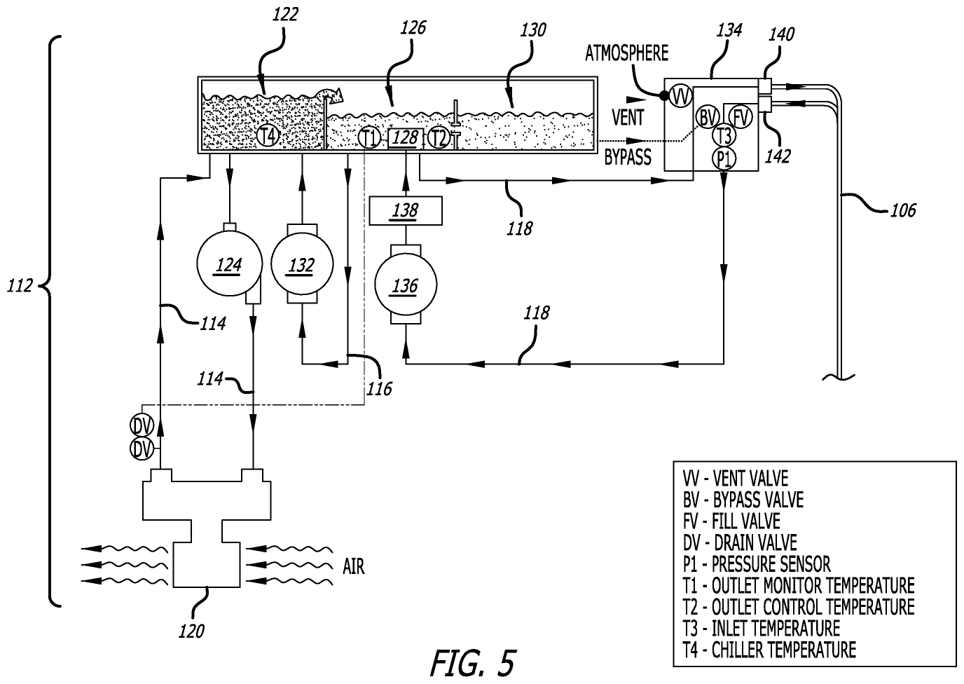

FIG. 5 illustrates a hydraulic system 112 of the control module 102 in accordance with some embodiments.

The control module 102 can also include the hydraulic system 112, which can include a chiller circuit 114, a mixing circuit 116, and a circulating circuit 118 for providing a temperature-controlled fluid.

The chiller circuit 114 can be configured for cooling a fluid (e.g., water, ethylene glycol, a combination of water and ethylene glycol, etc.) to produce a cooled fluid, which cooled fluid, in turn, can be for mixing with the mixed fluid in the mixing tank 126 set forth below to produce a supply fluid for TTM. The chiller circuit 114 can include a chiller evaporator 120 configured for the cooling of the fluid passing therethrough. The fluid for the cooling by the chiller evaporator 120 is provided by a chiller tank 122 using a chiller pump 124 of the chiller circuit 114.

The mixing circuit 116 can be configured for mixing spillover of the cooled fluid from the chiller tank 122 with a mixed fluid in a mixing tank 126 of the mixing circuit 116. The mixing circuit 116 can include a heater 128 in the mixing tank 126 configured for heating the mixed fluid to produce a heated fluid if needed for mixing with the cooled fluid to provide a supply tank 130 of the circulating circuit 118 with the supply fluid of a desired temperature for TTM. The mixing circuit 116 can include a mixing pump 132 configured to pump the fluid from the mixing tank 126 into the chiller tank 122 for producing the cooled fluid as well as the spillover of the cooled fluid for the mixing tank 126.

The circulating circuit 118 can be configured for circulating the supply fluid for TTM, which includes circulating the supply fluid provided by a manifold 134 through the one-or-more pads 104 using a circulation pump 136 directly or indirectly governed by a flow meter 138 of the circulating circuit 118. The manifold 134 can include an outlet 140 configured for discharging the supply fluid (e.g., a cooled fluid or a warmed fluid as indicated) from the hydraulic system 112 and an inlet 142 configured for charging the hydraulic system 112 with return fluid from the one-or-more pads 104 to continue to produce the supply fluid.

The primary FDL 106 can include primary tubing 144 configured to convey the supply fluid from the hydraulic system 112 by way of a lumen of the primary tubing 144 when fluidly connected thereto. Likewise, the primary tubing 144 is configured convey the return fluid back to the hydraulic system 112 by way of the lumen of the primary tubing 144 when fluidly connected thereto.

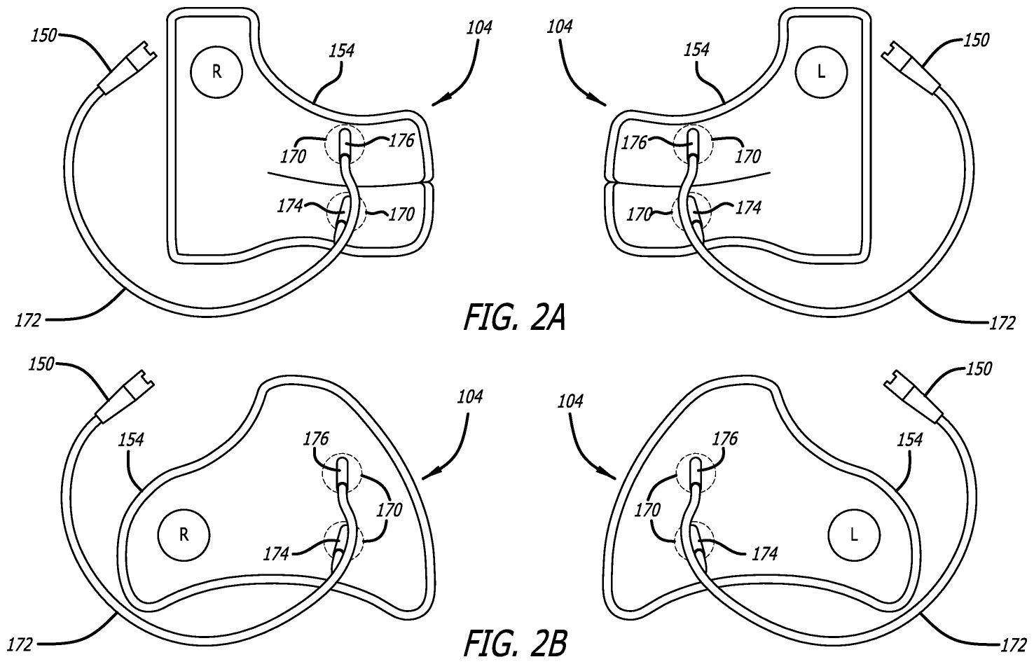

The primary tubing 144 of the primary FDL 106 of FIG. 2B can include a pair of opposing connector ends 146, wherein each connector end of the pair of connector ends 146 includes a corresponding primary FDL connector. For example, a connector end of the pair of connector ends 146 can include a primary FDL connector 148 configured to fluidly connect to a control-module connector (not shown) of the control module 102. Likewise, another connector end of the pair of connector ends 146 can include another primary FDL connector (not shown) configured to fluidly connect to a secondary FDL connector 150 of the one-or-more secondary FDLs 108.

Being as the one-or-more secondary FDLs 108 correspond with the one-or-more pads 104, description for the one-or-more secondary FDLs 108 is set forth in the following section.

### TTM Pads

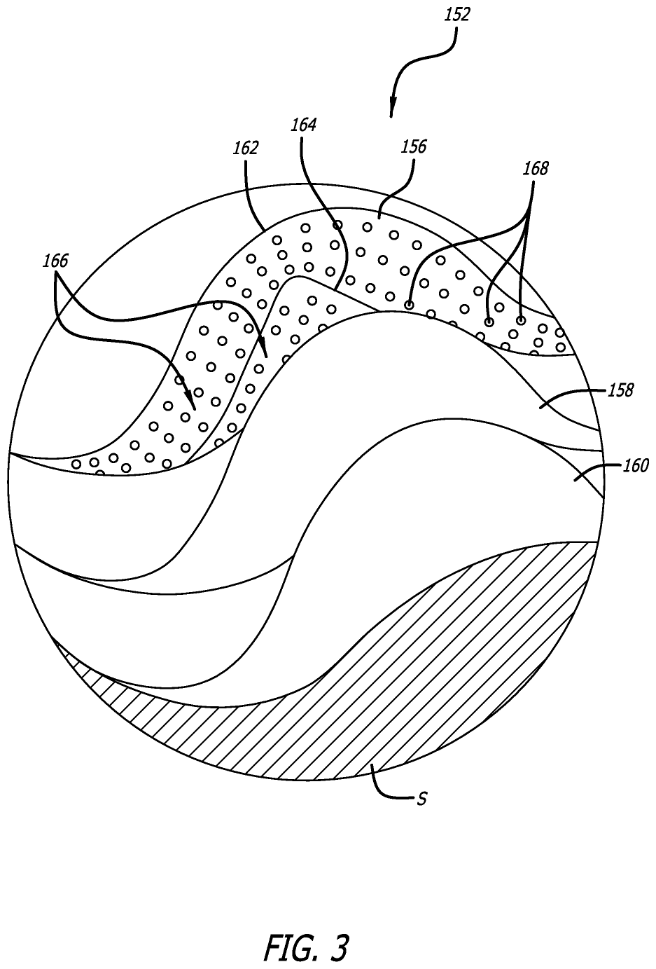

FIGS. 2A and 2B illustrate left and right pads of the one-or-more pads 104 respectively for a torso and legs of a patient in accordance with some embodiments. However, the one-or-more pads 104 can be configured for placement on any one or more portions of a patient's body, respectively, not just the torso or legs. FIG. 3 illustrates a multilayered pad body 152 of a pad of the-one-or-more pads 104 in accordance with some embodiments.

A pad of the one-or-more pads 104 can include the pad body 152, a release liner 154 over the pad body 152, and an indicator integrated into the pad body configured to visually indicate charging the pad with the supply fluid for confirmation of an accurate fluid connection to the control module 102.

The pad body 152 can include a conduit layer 156, an impermeable film 158 over the conduit layer 156, and a thermally conductive adhesive layer 160 over the impermeable film 158.

The conduit layer 156 can include a perimetrical wall 162 and one or more inner walls 164 extending from the conduit layer 156 toward the impermeable film 158. Together with the impermeable film 158, the perimetrical wall 162 and the one-or-more inner walls 164 form one or more conduits 166 configured to convey the supply fluid through the conduit layer 156. Optionally, the one-or-more conduits 166 can include one or more coatings thereon to promote internal reflection of any light directed into the one-or-more conduits 166. For example, the one-or-more conduits 166 can include a reflective coating thereon to promote internal reflection of any light directed into the one-or-more conduits 166. In another example, the one-or-more conduits 166 can include a coating or a pair of coatings thereon having a lower refractive index than the supply fluid to promote total internal reflection of any light directed into the one-or-more conduits 166.

The conduit layer 156 can include a plurality of protrusions 168 extending from the conduit layer 156 toward the impermeable film 158. The protrusions 168 can be configured to promote even flow of the supply fluid when the supply fluid is conveyed through the conduit layer 156. In accordance with the option of the one-or-more conduits 166 including the one-or-more coatings thereon to promote internal reflection of light, the protrusions 168 can also include the one-or-more coatings thereon to promote internal reflection of any light directed into the one-or-more conduits 166.

The conduit layer 156 can be a unitary piece of an opaque polymer (e.g., foam) including one or more translucent windows 170. For example, the one-or-more translucent windows 170 can be about (e.g., proximate, around, etc.) the pad inlet connector (not shown), the pad outlet connector (not shown), or both the pad inlet connector and the pad outlet connector set forth below. (The pad inlet and outlet connectors can be respectively inferred from the secondary FDL outlet connector 176 and the secondary FDL inlet connector 174 thereover in FIGS. 2A and 2B.) The one-or-more translucent windows 170 can be of a translucent polymer, thereby enabling visualization of the indicator with the charging of the conduit layer 156 with the supply fluid or the discharging of the conduit layer 156 with the return fluid.

Alternatively, the conduit layer 156 can be a unitary piece of translucent polymer such as an elastomer (e.g., silicone). Being that an entirety of such a conduit layer is translucent, the translucent elastomer, itself, enables visualization of the indicator with the charging of the conduit layer 156 with the supply fluid or the discharging of the conduit layer 156 with the return fluid.

Alternatively or additionally to the foregoing conduit layer 156 being of the opaque or translucent polymer, the pad inlet connector (not shown), the pad outlet connector (not shown), or both the pad inlet connector and the pad outlet connector set forth below can be translucent or include a translucent portion. (The pad inlet and outlet connectors can be respectively inferred from the secondary FDL outlet connector 176 and the secondary FDL inlet connector 174 thereover in FIGS. 2A and 2B.) When translucent, the pad inlet connector or the pad outlet connector enables visualization of the indicator with the charging of the conduit layer 156 with the supply fluid.

The impermeable film 158 can be configured to retain the supply fluid in the conduit layer 156 when the supply fluid is conveyed through the conduit layer 156. In addition, the impermeable film 158 can be configured to allow efficient energy transfer between the conduit layer 156 and the adhesive layer 160.

The adhesive layer 160 can be configured for placement on skin S (see FIG. 3) of a portion (e.g., torso, leg, etc.) of a patient's body for direct thermal conduction through the adhesive layer 160. While the adhesive layer 160 can be configured to conformably adhere to the portion of the patient's body for better thermal conduction, adherence of the adhesive layer 160 to the portion of the patient's body can be optimized to avoid irritating or wounding the portion of the patient's body upon removal of the pad.

The adhesive layer 160 can include a hydrogel or hydrogel matrix. The hydrogel can be selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

The release liner 154 can be over the adhesive layer 160 in a ready-to-use state of the pad. The release liner 154 can be configured to maintain integrity of at least the adhesive layer 160 prior to use of the pad.

In addition to the foregoing, the pad can include an inlet connector (under the secondary FDL outlet connector 176 in FIGS. 2A and 2B) and an outlet connector (under the secondary FDL inlet connector 174 in FIGS. 2A and 2B). The inlet connector can be configured for charging the conduit layer 156 with the supply fluid, while the outlet connector can be configured for discharging the return fluid from the conduit layer 156.

A pad of the one-or-more pads 104 can include a secondary FDL of the one-or-more secondary FDLs 108. For example, the secondary FDL can be pre-connected to the pad as sold.

The secondary FDL can include secondary tubing 172 configured to convey the supply fluid from the primary FDL 106 when connected to the hydraulic system 112 of the control module 102. Likewise, the secondary tubing 172 can be configured to convey the return fluid back to the primary FDL 106 when connected to the hydraulic system 112 of the control module 102.

The secondary FDL can be split at a pad-connecting end of the secondary FDL. The pad-connecting end of the secondary FDL can include a pair of secondary FDL connectors. A secondary FDL inlet connector 174 of the pair of secondary FDL connectors can be configured to fluidly connect to the outlet connector of the pad. A secondary FDL outlet connector 176 of the pair of secondary FDL connectors can be configured to fluidly connect to the inlet connector of the pad.

At least a portion of the secondary tubing 172 up to an entirety of the secondary tubing 172 can be translucent. The translucent portion of the secondary tubing 172 or the entirety of the secondary tubing 172 when translucent enables visualization of the indicator with the charging of the conduit layer 156 with the supply fluid.

Alternatively or additionally, the secondary FDL outlet connector 176, the secondary FDL inlet connector 174, or both the secondary FDL outlet connector 176 and the secondary FDL inlet connector 174 can be translucent or include a translucent portion. When translucent, the secondary FDL outlet connector 176 or the secondary FDL inlet connector 174 enables visualization of the indicator with the charging of the conduit layer with the supply fluid.

The indicator for confirming fluid connections can be a chemical indicator disposed in the one-or-more conduits 166 or integrated into the conduit layer 156 itself.

The chemical indicator can be a salt such as CoCl₂ disposed in the one-or-more conduits 166. The CoCl₂ can be provided in an amount sufficient to impart a discernable pink color (i.e., the color of CoCl₂.6H₂O) to the supply fluid when solvated in the supply fluid with the charging of the conduit layer 156 with the supply fluid.

Alternatively, the chemical indicator can be a fluorophore disposed in the one-or-more conduits 166. Like the foregoing salt, the fluorophore can be provided in an amount sufficient to impart a discernable fluorescence to the supply fluid when solvated in the supply fluid with the charging of the conduit layer 156 with the supply fluid. The fluorophore can be an oxidant or combined with an oxidant in some embodiments such that a portion of the amount provided in the one-or-more conduits 166 can oxidize and therefore sterilize that which it comes into contact with.

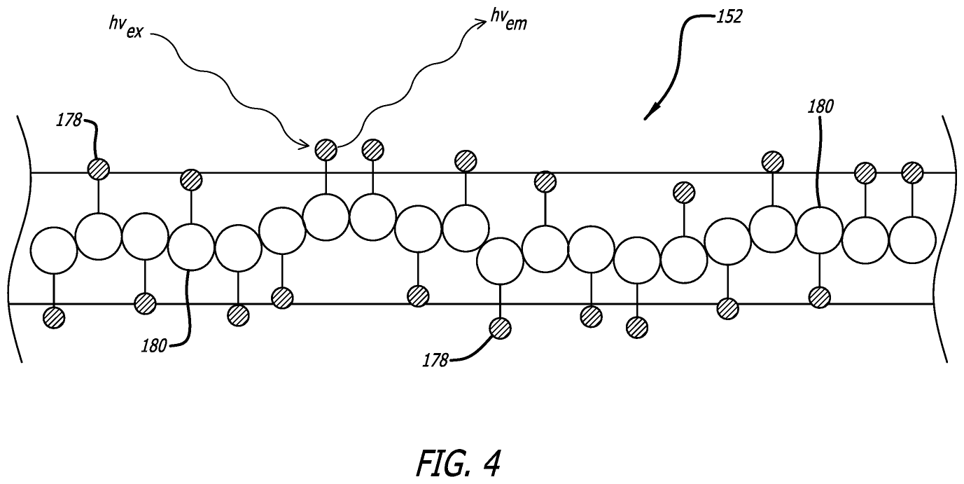

FIG. 4 illustrates a fluorophore covalently bonded to a translucent polymer in accordance with some embodiments.

In an alternative to disposing the fluorophore in the one-or-more conduits 166, the fluorophore can be covalently bonded as a fluorescent moiety to the translucent polymer forming the conduit layer 156. For example, the fluorescent moiety can be pendant fluorescent group 178 of a monomer residue 180 of the translucent polymer. The fluorophore can be provided in an amount sufficient to impart a discernable fluorescence to the supply fluid when partially solvated in the supply fluid with the charging of the conduit layer 156 with the supply fluid. Advantageously, the fluorophore, being a fluorescent moiety of the translucent polymer, remains bonded to the translucent polymer such that it does not contaminate the supply fluid.

Whether disposed in the one-or-more conduits 166 or covalently bonded to the translucent polymer, the fluorophore can be configured to absorb light about an excitation wavelength and emit light about an emission wavelength different than the absorption wavelength. (See, for example, hvₑₓ and hvₑₘ in FIG. 4.) While it is advantageous for both absorption and emission wavelengths to be in the visible portion of the electromagnetic spectrum, at least the emission wavelength is in the visible portion (e.g., 380 nm-750 nm) of the electromagnetic spectrum. In addition, the fluorophore can exhibit solvatochromism in accordance with a composition of the supply fluid, which can be water, ethylene glycol, or a mixture thereof as set forth above. The fluorophore can exhibit a hypsochromic shift (e.g., a blue shift) from a more to less polar supply fluid or a bathochromic shift (e.g., a red shift) from the more to less polar supply fluid. Said differently, the fluorophore can exhibit a bathochromic shift (e.g., a red shift) from a less to more polar supply fluid or a hypsochromic shift (e.g., a blue shift) from the less to more polar supply fluid. Such a feature is useful for optimizing the indicator under different lighting conditions.

Instead of the chemical indicator, the indicator for confirming fluid connections can be an integrated LED module disposed in at least the secondary FDL outlet connector 176. The LED module can include one or more LEDs configured to illuminate the supply fluid as it flows through the secondary FDL outlet connector 176. In order to power the one-or-more LEDs, the LED module can include a water turbine and an electrical generator. The water turbine can be configured to rotate a rotor of the electrical generator relative to a stator of the electrical generator to produce electrical energy to illuminate the supply fluid with the one-or-more LEDs. Advantageously, the LED module can include a temperature sensor and LED-illuminating logic. The LED-illuminating logic can be configured to selectively illuminate one LED of the one-or-more LEDs in accordance with a predetermined temperature range. Each LED of the one-or-more LEDs can be configured to illuminate the supply fluid with a different color (e.g., blue for cooler temperature range, red for warmer temperature range, and green for a temperature therebetween).

The indicator for confirming fluid connections can alternatively be an integrated laser diode module disposed in at least the secondary FDL outlet connector 176. The laser diode module can include one or more laser diodes configured to illuminate the supply fluid as it flows through the secondary FDL outlet connector 176. Advantageously, when the one-or-more conduits 166 and the plurality of protrusions 168 include the one-or-more coatings thereon to promote internal reflection of light, the light from the one-or-more laser diodes can be piped through the conduit layer 156, thereby illuminating an entirety of the conduit layer 156. In order to power the one-or-more laser diodes, the laser diode module, like the LED module, can include a water turbine and an electrical generator. The water turbine can be configured to rotate a rotor of the electrical generator relative to a stator of the electrical generator to produce electrical energy to illuminate the supply fluid with the one-or-more laser diodes. Advantageously, the laser diode module can include a temperature sensor and laser diode-illuminating logic. The laser diode-illuminating logic can be configured to selectively illuminate one laser diode of the one-or-more laser diodes in accordance with a predetermined temperature range. Each laser diode of the one-or-more laser diodes can be configured to illuminate the supply fluid with a different color (e.g., blue for cooler temperature range, red for warmer temperature range, and green for a temperature therebetween).

### Methods

Methods of the systems and pads include methods of use. For example, a method of using the system 100 can include one or more steps selected from a release liner-removal step, a pad-placing step, a pad-connecting step, a charging step, a confirming step, and a fluid-circulating step.

The release liner-removal step can include removing the release liner 154 of a pad of the one-or-more pads 104 to reveal the adhesive layer 160 before the pad-placing step. As set forth above, the release liner 154 is configured to maintain integrity of at least the adhesive layer 160 prior to using the pad.

The pad-placing step can include placing the pad on a portion of a patient's body with the adhesive layer 160 of the pad in contact with skin S (see FIG. 4) of the portion of the patient's body.

If a secondary FDL of the one-or-more secondary FDLs 108 is not connected to the foregoing pad of the one-or-more pads 104, the method can further include a pad-connecting step. The pad-connecting step can include fluidly connecting the secondary FDL outlet connector 176 at the split pad-connecting end of the secondary FDL to a pad inlet connector. The pad-connecting step can also include fluidly connecting the secondary FDL inlet connector 174 at the split pad-connecting end of the secondary FDL to a pad outlet connector.

The charging step can include charging the conduit layer 156 of the pad with the supply fluid. As set forth above, the supply fluid can be provided by the hydraulic system 112 of the control module 102 by way of a combination of fluidly connected FDLs including the secondary FDL and the primary FDL 106.

The confirming step can include visually confirming a presence of the indicator, which, as set forth above, can be configured to visually indicate the charging of the conduit layer 156 with the supply fluid for confirmation of an accurate fluid connection.

The confirming step can include confirming a color or fluorescence of the chemical indicator solvated in the supply fluid. Such confirming can be through a translucent window of the one-or-more windows 170 of the pad body 152, a translucent polymer of the pad body 152, a translucent portion of the secondary FDL, or a translucent portion of one or more connectors of the pad inlet connector, the pad outlet connector, the secondary FDL outlet connector 176, and the secondary FDL inlet connector 174.

The confirming step can include confirming illumination of the supply fluid with the integrated LED module of at least the secondary FDL outlet connector 176 through a translucent window of the one-or-more windows 170 of the pad body 152, a translucent polymer of the pad body 152, a translucent portion of the secondary FDL, or a translucent portion of one or more connectors of the pad inlet connector, the pad outlet connector, the secondary FDL outlet connector 176, and the secondary FDL inlet connector 174.

Following on the confirming step with the integrated LED, the method can further include another confirming step. The other confirming step can include visually confirming the supply fluid is within a particular temperature range. As set forth above, the LED module can include the temperature sensor and the LED-illuminating logic configured to selectively illuminate one LED of one or more LEDs in accordance with a predetermined temperature range. Each LED of the one-or-more LEDs can be configured to illuminate the supply fluid with a different color.

The fluid-circulating step can include circulating the supply fluid through the conduit layer 156 of the pad body 152 to cool or warm the portion of the patient's body as needed in accordance with TTM. Indeed, the fluid-circulating step can include transferring heat between the supply fluid and the portion of the patient's body by thermal conduction through the adhesive layer 160. For example, the fluid-circulating step can include circulating a cool fluid through the conduit layer 156 to bring the patient into hypothermia. The fluid-circulating step can include circulating a warm fluid through the conduit layer 156 to bring the patient into normothermia.

While the method set forth above is described with reference to a single pad of the one-or-more pads 104, it should be understood that any number of pads of the one-or-more pads 104 can be used as necessary to effectuate a desired treatment by way of TTM.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A pad for targeted temperature management (“TTM”), comprising:
a multilayered pad body including:
a conduit layer including one or more conduits configured to convey a temperature-controlled fluid as a supply fluid from a hydraulic system of a control module and convey a return fluid back to the hydraulic system; and
a thermally conductive adhesive layer over the conduit layer configured for placement on a portion of a patient's body;
a pad inlet connector including a pad inlet configured for charging the conduit layer with the supply fluid;
a pad outlet connector including a pad outlet configured for discharging the return fluid from the conduit layer; and
an indicator configured to visually indicate the charging of the conduit layer with the supply fluid for confirmation of an accurate fluid connection.

2. The pad of claim 1, the pad body further comprising one or more translucent windows of the pad body enabling visualization of the indicator with the charging of the conduit layer with the supply fluid.

3. The pad of claim 2, wherein the one-or-more translucent windows are about the pad inlet connector, the pad outlet connector, or both the pad inlet connector and the pad outlet connector.

4. The pad of claim 1, wherein the pad body is of a translucent elastomer enabling visualization of the indicator with the charging of the conduit layer with the supply fluid.

5. The pad of claim 1, further comprising a secondary line (“FDL”) configured to convey the supply fluid from the hydraulic system and convey the return fluid back to the hydraulic system, the secondary FDL split at a pad-connecting end of the secondary FDL, and the pad-connecting end of the secondary FDL including a pair of secondary FDL connectors including a secondary FDL outlet connector configured to fluidly connect to the pad inlet connector and a secondary FDL inlet connector configured to fluidly connect to the pad outlet connector.

6. The pad of claim 5, wherein at least a portion of the secondary FDL is translucent, thereby enabling visualization of the indicator with the charging of the conduit layer with the supply fluid.

7. The pad of claim 5, wherein one or more connectors of the pad inlet connector, the pad outlet connector, the secondary FDL outlet connector, and the secondary FDL inlet connector include a translucent portion, thereby enabling visualization of the indicator with the charging of the conduit layer with the supply fluid.

8. The pad of claim 1, wherein the indicator is a chemical indicator disposed in the one-or-more conduits or integrated into the conduit layer.

9. The pad of claim 8, wherein the chemical indicator is CoCl₂ disposed in the one-or-more conduits, the CoCl₂ provided in an amount sufficient to impart a discernable pink color to the supply fluid when solvated in the supply fluid with the charging of the conduit layer with the supply fluid.

10. The pad of claim 8, wherein the chemical indicator is a fluorophore disposed in the one-or-more conduits, the fluorophore provided in an amount sufficient to impart a discernable fluorescence to the supply fluid when solvated in the supply fluid with the charging of the conduit layer with the supply fluid.

11. The pad of claim 8, wherein the chemical indicator is a fluorophore covalently bonded to a polymer forming the conduit layer, the fluorophore provided in an amount sufficient to impart a discernable fluorescence to the supply fluid when partially solvated in the supply fluid with the charging of the conduit layer with the supply fluid.

12. The pad of claim 10, wherein the fluorophore absorbs light about an excitation wavelength and emits light about an emission wavelength, at least the emission wavelength being in a visible portion of the electromagnetic spectrum.

13. The pad of claim 11, wherein the fluorophore exhibits solvatochromism in accordance with a composition of the supply fluid, the supply fluid being water, ethylene glycol, or a mixture thereof.

14. The pad of claim 5, wherein at least the secondary FDL outlet connector includes an integrated light-emitting diode (“LED”) module including one or more LEDs configured to illuminate the supply fluid as it flows through the secondary FDL outlet connector.

15. The pad of claim 14, wherein the LED module includes a water turbine and an electrical generator, the water turbine configured to rotate a rotor of the electrical generator relative to a stator of the electrical generator to produce electrical energy to illuminate the supply fluid with the one-or-more LEDs.

16. The pad of claim 14, wherein the LED module includes a temperature sensor and LED-illuminating logic configured to selectively illuminate one LED of the one-or-more LEDs in accordance with a predetermined temperature range, each LED of the one-or-more LEDs configured to illuminate the supply fluid with a different color.

17. The pad of claim 5, wherein at least the secondary FDL outlet connector includes an integrated laser diode module including one or more laser diodes configured to illuminate the supply fluid as it flows through the secondary FDL outlet connector, the one-or-more conduits configured to promote internal reflection of light and illuminate an entirety of the conduit layer.

18. The pad of claim 1, further comprising an impermeable film between the conduit layer and the adhesive layer configured to retain the supply fluid in the conduit layer.

19. The pad of claim 1, wherein the adhesive layer includes a hydrogel selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels.

20. The pad of claim 1, further including a release liner over the adhesive layer in a ready-to-use state of the pad, the release liner configured to maintain integrity of at least the adhesive layer prior to use of the pad.

21. A system for targeted temperature management (“TTM”), comprising:
a control module including a hydraulic system configured to provide a temperature-controlled fluid;
a primary fluid delivery line (“FDL”) configured to convey the temperature-controlled fluid as a supply fluid from the hydraulic system and convey a return fluid back to the hydraulic system; and
one or more pads configured for placement on one-or-more portions of a patient's body, respectively, each pad of the one-or-more pads including:
a multilayered pad body including a conduit layer having one or more conduits configured to convey the temperature-controlled fluid;
a pad inlet connector including a pad inlet configured for charging the conduit layer with the supply fluid;
a pad outlet connector including a pad outlet configured for discharging the return fluid from the conduit layer; and
an indicator configured to visually indicate the charging of the conduit layer with the temperature-controlled fluid for confirmation of an accurate fluid connection.

22. The system of claim 21, the hydraulic system further including:
a chiller evaporator configured for fluid cooling;
a heater configured for fluid heating, the chiller evaporator and the heater, together, configured to provide the temperature-controlled fluid;
a hydraulic-system outlet configured for discharging the supply fluid from the hydraulic system; and
a hydraulic-system inlet configured for charging the hydraulic system with the return fluid to continue to produce the temperature-controlled fluid.

23. The system of claim 21, the control module further including one or more processors, primary memory, and instructions stored in the primary memory configured to instantiate one or more processes for TTM with the control module.

24-32. (canceled)
